(19) United States
(12) Patent Application Publication
(10) Pub. No.: US 2016/0213886 A1
(43) Pub. Date: Jul. 28, 2016

(54) DRAIN TUBE STABILIZING BELT

(71) Applicant: Paul L. DiGiorgi, Fort Myers, FL (US)

(72) Inventor: Paul L. DiGiorgi, Fort Myers, FL (US)

(21) Appl. No.: 14/604,707

(22) Filed: Jan. 24, 2015

(51) Int. Cl. A61M 25/02; A61F 5/44; A61M 27/00

(52) U.S. Cl. CPC A61M 25/02; A61M 27/00; A61F 5/4408; A61M 2025/0206; A61M 2025/0233

(57) ABSTRACT

A drain tube stabilizing belt (1) that wraps around a patient's abdomen and has locking straps (13) located thereon for securing excess drain tubes (12) in place to prevent pulling on the patient's incisions. The drain tube stabilizing belt preferably has one or more loops (16) and/or pouches (21) located thereon for holding for holding drain bulbs, EKG monitors, pacemakers and/or other devices.

### FIELD OF THE INVENTION

This invention relates to post-surgery garments and devices used to increase patient comfort and, more particularly, a belt that is worn around a patient's abdomen that locks one or more drain tubes into place on an incision, thereby preventing the drain tubes from shifting when the patient moves.

### BACKGROUND OF THE INVENTION

Surgical drains are tubes placed in incision sites by surgeons after surgery to remove pus, blood and/or other fluids that accumulate in the wound. While drain tubes are often medically necessary, they can be a major source of discomfort for the patient. This is especially true if the drain tube is placed in a sensitive area near nerves, such as between a patient's ribs after heart or lung surgery.

A further problem occurs with other devices attached to the patient, such as drain bulbs, EKG monitors, pacemakers and so forth. These devices can shift and become tangled with the drain tubes or fall, thereby compromising patient safety and comfort.

Therefore, a need exists for a drain tube stabilizing belt that locks one or more drain tubes into place on an incision, thereby preventing the drain tubes from shifting when the patient moves. In addition, a need exists for a drain tube stabilizing belt that provides storage for devices, such as drain bulbs, EKG monitors, pacemakers and so forth, so these devices do not shift and move easily with the patients.

The relevant prior art includes the following references:

| | | |
|---|---|---|
| Patent No. | Inventor | Issue/Publication Date |

(U.S. Patent References)

| | | |
|---|---|---|
| 4,445,894 | Kovacs | May 1, 1984 |
| 5,271,745 | Fentress et al. | Dec. 21, 1993 |
| 5,624,403 | Jaquith | Apr. 29, 1997 |
| 6,125,915 | Watson et al. | Nov. 28, 2000 |
| 6,610,032 | Prody | Aug. 26, 2003 |
| 6,645,185 | Bird et al. | Nov. 11, 2003 |
| 8,066,657 | Frazer | Nov. 29, 2011 |

(Foreign Patent References)

| | | |
|---|---|---|
| CN202605503 | N/A | Dec. 19, 2012 |

### SUMMARY OF THE INVENTION

The primary object of the present invention is to provide a drain tube stabilizing belt that locks one or more drain tubes into place on an incision, thereby preventing the drain tubes from shifting when the patient moves.

An additional object of the present invention is to provide a drain tube stabilizing belt that provides storage for devices, such as drain bulbs, EKG monitors, pacemakers and so forth, so these devices do not shift and become tangled with drain tubes, thereby pulling on the drain tubes and compromising patient safety and comfort.

The present invention fulfills the above and other objects by providing a drain tube stabilizing belt that wraps around a patient's abdomen and has locking straps located thereon for securing drain tubes in place to prevent pulling on the patient's incisions. The drain tube stabilizing belt preferably has one or more loops and/or pouches located thereon for holding for holding drain bulbs, EKG monitors, pacemakers and/or other devices.

The drain tube stabilizing belt of the present invention is preferably constructed from an elastomeric material that will fit snugly around a patient's abdomen while still allowing the patient to breath. A hook and loop and fastener is preferably used for attaching the drain tube stabilizing belt around the abdomen of the patient so that the belt may be easily adjusted.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a drain tube stabilizing belt of the present invention.

FIG. 2 is a front view of a drain tube stabilizing belt of the present invention in use; and

FIG. 3 is a rear view of the drain tube stabilizing belt of the present invention in use.

### DESCRIPTION OF THE PREFERRED EMBODIMENTS

For purposes of describing the preferred embodiment, the terminology used in reference to the numbered components in the drawings is as follows:
1. drain tube stabilizing belt, generally
2. body
3. top edge
4. bottom edge
5. first end
6. second end
7. front surface
8. rear surface
9. attachment means
10. hook and loop fastener
11. locking means
12. drain tube
13. locking strap
14. male end
15. female end
16. loop
17. drain bulb
18. aperture
19. front surface of male end
20. front surface of female end
21. pouch
22. medical device

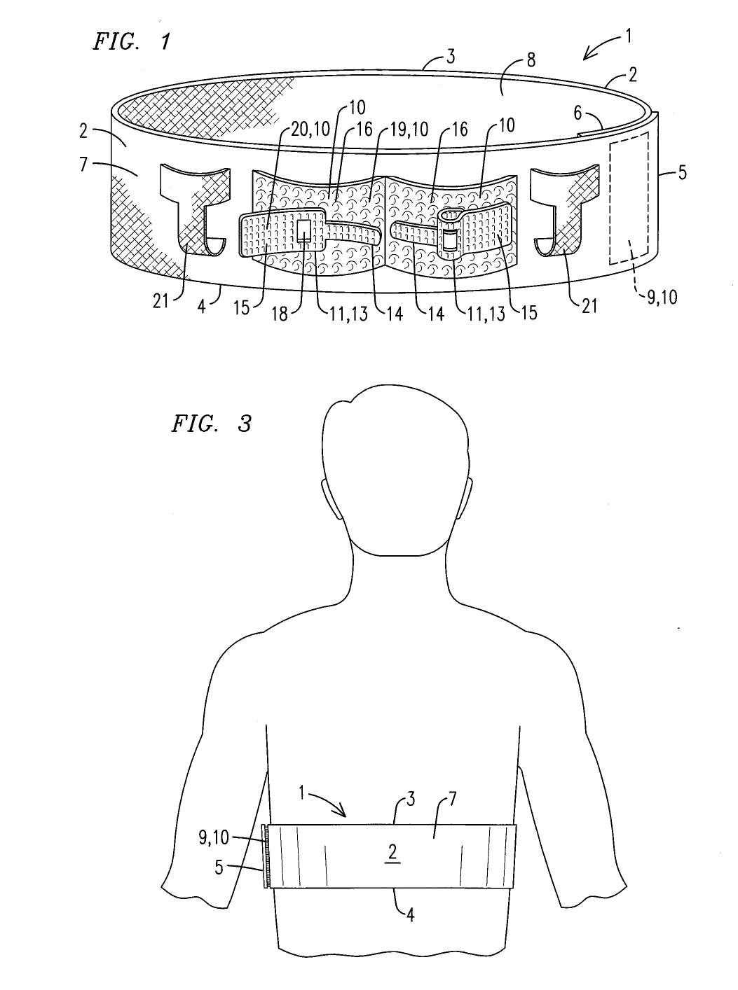

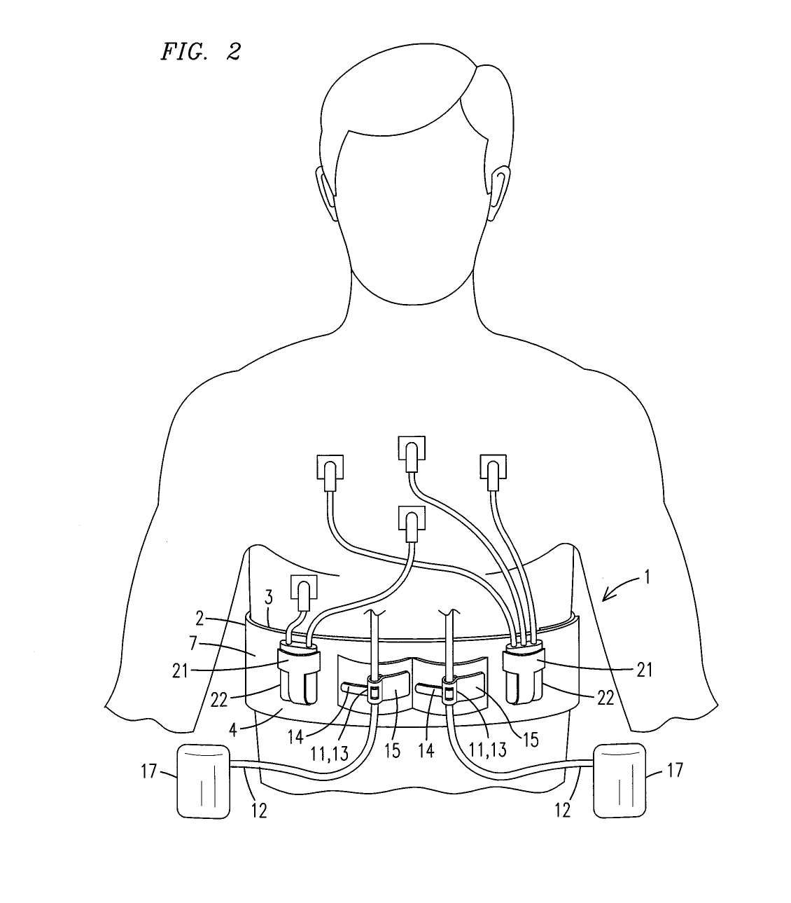

With reference to FIGS. 1-3, a front perspective view of a drain tube stabilizing belt 1 of the present invention, a front view of a drain tube stabilizing belt 1 of the present invention in use and a rear view of the drain tube stabilizing belt 1 of the present invention in use, respectively, are illustrated. The drain tube stabilizing belt 1 comprises an elongated rectangular-shaped main body 2 having a top edge 3, a bottom edge 4, a first end 5, a second end 6, a front surface 7 and a rear surface 8. The drain tube stabilizing belt 1 wraps around a patient's abdomen with the rear surface 8 facing the patient's skin and is secured in place with an attachment means 9, such as a hook and loop fastener 10 at each end.

At least one locking means 11 is located on the front surface of the drain tube stabilizing belt 1 and used for securing drain tubes 12 in place to prevent pulling on the patient's incisions. The locking means 11 may be locking straps 13 with hook and loop fasteners 10, clips or other means for securing one or more drain tubes 12 to the front surface 7 of the drain tube stabilizing belt 1. As illustrated here, the locking straps 13 comprise a male end 14 and a female end 15 secured to a hook and loop fastener 10 located on a loop 16 used for securing a drain bulb 17 to the drain tube stabilizing belt 1. The locking strap 13 is secured to the hook and loop fastener 10 along a central axis, thereby allowing the male end 14 to be folded over a drain tube 12 and placed through an aperture 18 located on the female end 15. The male end 14 is the secured to the hook and loop fastener 10 located on the loop 16 by hook and loop fastener 10 located on a front surface 19 of the male end 14. Then, the female end 15 is folded over and secured to the hook and loop fastener 10 located on the loop 16 by hook and loop fastener 10 located on the front surface 20 of the female end 15, thereby wrapping the locking strap 13 around the drain tube 12.

The drain tube stabilizing belt 1 preferably comprises loops 16 and/or pouches 21 located on the front surface 7 of the drain tube stabilizing belt 1 for holding medical devices 22, such as drain bulbs, EKG monitors, pacemakers and/or other devices. These, loops 16 and/or pouches 21 keep wires and drain tubes 12 extending from the medical devices 22 organized and prevent the medical devices 22 from falling and becoming tangled and/or pulling on each other.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

Having thus described my invention, I claim:

1. A drain tube stabilizing belt comprising:
an elongated substantially rectangular-shaped main body having a top edge, a bottom edge, a first end, a second end, a front surface and a rear surface;
an attachment means for securing located proximal to the first end of the drain tube stabilizing belt for securing the drain tube stabilizing belt in place around a patient's abdomen with the rear surface of the drain tube stabilizing belt facing the patient's skin; and
at least one locking means located on the front surface of the drain tube stabilizing belt used for securing drain tubes in place to prevent pulling on the patient's incisions.

2. The drain tube stabilizing belt of claim 1 further comprising:
at least one loop located on a front surface of the drain tube stabilizing belt for holding a medical device.

3. The drain tube stabilizing belt of claim 1 further comprising:
at least one pouch located on a front surface of the drain tube stabilizing belt for holding a medical device.

4. The drain tube stabilizing belt of claim 1 wherein:
said at least one locking means is at least one locking strap.

5. The drain tube stabilizing belt of claim 1 wherein:
said at least one locking means is at least one clip.

6. The drain tube stabilizing belt of claim 2 wherein:
said at least one locking means is located on the least one loop located on the front surface of the drain tube stabilizing belt.

7. The drain tube stabilizing belt of claim 3 wherein:
said at least one locking means is located on at least one pouch located on the front surface of the drain tube stabilizing belt.

8. The drain tube stabilizing belt of claim 4 wherein:
said at least one locking strap comprises a male end and a female end secured to a hook and loop fastener located proximal to a front surface of the tube stabilizing belt;
said locking strap is secured to the hook and loop fastener along a central axis, thereby allowing the male end to be folded over a drain tube and placed through an aperture located on the female end;
said male end having hook and loop fastener located on a front surface of the male end; and
said female end having hook and loop fastener located on a front surface of the female end.

9. A drain tube stabilizing belt comprising:
an elongated rectangular-shaped main body having a top edge, a bottom edge, a first end, a second end, a front surface and a rear surface;
an attachment means for securing located proximal to the first end of the drain tube stabilizing belt for securing the drain tube stabilizing belt in place around a patient's abdomen with the rear surface of the drain tube stabilizing belt facing the patient's skin; and
at least one locking strap located on the front surface of the drain tube stabilizing belt used for securing drain tubes in place to prevent pulling on the patient's incisions.

10. The drain tube stabilizing belt of claim 9 further comprising:
at least one loop located on a front surface of the drain tube stabilizing belt for holding a medical device.

11. The drain tube stabilizing belt of claim 9 further comprising:
at least one pouch located on a front surface of the drain tube stabilizing belt for holding a medical device.

12. The drain tube stabilizing belt of claim 10 wherein:
said at least one locking strap is located on the at least one loop located on the front surface of the drain tube stabilizing belt.

13. The drain tube stabilizing belt of claim 11 wherein:
said at least one locking strap is located on at least one pouch located on the front surface of the drain tube stabilizing belt.

14. The drain tube stabilizing belt of claim 9 wherein:
said at least one locking strap comprises a male end and a female end secured to a hook and loop fastener located proximal to a front surface of the tube stabilizing belt;
said locking strap is secured to the hook and loop fastener along a central axis, thereby allowing the male end to be folded over a drain tube and placed through an aperture located on the female end;
said male end having hook and loop fastener located on a front surface of the male end; and
said female end having hook and loop fastener located on a front surface of the female end.

15. A drain tube stabilizing belt comprising:
an elongated rectangular-shaped main body having a top edge, a bottom edge, a first end, a second end, a front surface and a rear surface;
an attachment means for securing located proximal to the first end of the drain tube stabilizing belt for securing the drain tube stabilizing belt in place around a patient's abdomen with the rear surface of the drain tube stabilizing belt facing the patient's skin;
at least one locking strap located on the front surface of the drain tube stabilizing belt used for securing drain tubes in place to prevent pulling on the patient's incisions;
said at least one locking strap comprises a male end and a female end secured to a hook and loop fastener located proximal to a front surface of the tube stabilizing belt;
said locking strap is secured to the hook and loop fastener along a central axis, thereby allowing the male end to be folded over a drain tube and placed through an aperture located on the female end;
said male end having hook and loop fastener located on a front surface of the male end; and
said female end having hook and loop fastener located on a front surface of the female end.

16. The drain tube stabilizing belt of claim 9 further comprising:
at least one loop located on a front surface of the drain tube stabilizing belt for holding a medical device.

17. The drain tube stabilizing belt of claim 9 further comprising:
at least one pouch located on a front surface of the drain tube stabilizing belt for holding a medical device.

18. The drain tube stabilizing belt of claim 10 wherein:
said at least one locking strap is located on the at least one loop located on the front surface of the drain tube stabilizing belt.

19. The drain tube stabilizing belt of claim 11 wherein:
said at least one locking strap is located on at least one pouch located on the front surface of the drain tube stabilizing belt.
